(19) United States
(12) Patent Application Publication
(10) Pub. No.: US 2015/0002814 A1
(43) Pub. Date: Jan. 1, 2015

(54) Microscopy System and Method for Imaging Phase Objects

(71) Applicants: Carl Zeiss AG, Oberkochen (DE); Carl Zeiss Meditec AG, Jena (DE)

(72) Inventors: Markus SEESSELBERG, Aalen (DE); Michael WICK, Munchen (DE); Christoph HAUGER, Aalen (DE); Holger MATZ, Unterschneidheim (DE); Jennifer-Magdalena MASCH, Gottingen (DE)

(21) Appl. No.: 14/319,050

(22) Filed: Jun. 30, 2014

(30) Foreign Application Priority Data
Jun. 28, 2013 (DE) 10 2013 010 844.6

(51) Int. Cl. A61B 3/13; G02B 7/28; A61B 3/00; A61B 3/14

(52) U.S. Cl. CPC A61B 3/13; A61B 3/14; G02B 7/28; A61B 3/0008
USPC 351/206; 351/246; 359/385; 351/221

(57) ABSTRACT

Disclosed is a microscope system for imaging an object within an eye, wherein the object is a phase object. The microscope system includes imaging optics for generating images of an object plane of the imaging optics in an image plane of the imaging optics by using imaging light. The object plane is optically conjugate to the image plane. The microscope system is configured to store focus shift data representing a focus shift. The focus shift is generated by a different effect on the imaging light generated by phase objects as compared to amplitude objects. The microscope system is configured to image the object depending on the focus shift data.

### CROSS-REFERENCES TO RELATED APPLICATIONS

The present application claims priority of Patent Application No. 10 2013 010 844.6, filed Jun. 28, 2013 in Germany, the entire contents of which are incorporated by reference herein.

### FIELD OF THE INVENTION

The present invention relates to a microscope system for imaging phase objects by bright field microscopy. Specifically, the present invention relates to a microscope system for determining the position of phase objects and/or for imaging phase objects, which are located at a given object distance.

### BACKGROUND ART

In the field of microscopy a distinction is commonly made between phase objects and amplitude objects. Phase objects alter the phase of the transmitted microscopy light, whereas the amplitude of the transmitted light is altered only to an insignificant extent. On the other hand, amplitude objects lower the intensity of the transmitted microscopy light to a significant extent.

Amplitude objects are usually imaged by using bright-field microscopy. For imaging phase objects, phase contrast microscopes and dark-field microscopes have been developed. Phase contrast microscopes and dark-field microscopes require a dark-field aperture or a phase-contrast aperture, which is arranged along the imaging beam path. The position of these apertures has to be finely adjusted. This, however, renders it difficult to quickly switch from a bright-field imaging mode to either a phase contrast imaging mode or a dark-field imaging mode. Moreover, it is typically necessary to adjust the position of the dark-field or phase contrast aperture depending on the selected magnification of the zoom system. Furthermore, it has proved to be difficult to combine a phase contrast microscope or a dark-field microscope with a red reflex illumination system for inspecting eyes, since the position of the phase contrast aperture or the dark-field aperture has to be adapted to the ametropia of the eye under inspection.

For imaging phase objects, procedures have been developed in which phase objects are stained with dye in order to allow imaging these objects as amplitude objects. However, these procedures are disadvantageous in that the dyes usually cannot be removed again from the object. This can make it impossible to inspect the same object by using further techniques. Furthermore, some of the dyes are toxic and can therefore not be applied to living biological material.

Accordingly, it is desirable to provide a microscope system and a method for operating a microscope system, which allows efficient imaging of phase objects.

Embodiments provide a microscope system for imaging an object within an eye, wherein the object is a phase object. The microscope system may comprise imaging optics for generating images of an object plane of the imaging optics in an image plane of the imaging optics by using imaging light. The object plane may be optically conjugate to the image plane. The microscope system may be configured to store focus shift data representing a focus shift, wherein the focus shift is generated by a different effect on the imaging light generated by phase objects as compared to amplitude objects. The microscope system may further be configured to image the object, depending on the focus shift data.

It has been shown that a focus shift occurs as a result of the fact that phase objects have a different effect on imaging light than amplitude objects. As a result of these different effects, phase objects and amplitude objects, which are located at a same object distance, are imaged by the imaging optics onto focus planes, which have different positions along the optical axis of the imaging optics.

It has further been shown that the focus shift can be characterized by focus shift data. Moreover, it has been shown that by storing the focus shift data and performing the imaging of the phase objects depending on the stored focus shift data, it is possible to efficiently image phase objects and/or to determine the position of phase objects relative to the microscope system.

Phase objects may be defined as objects, which alter the phase of the light, which is transmitted through the phase object, but which leave the amplitude of the transmitted light substantially unchanged. Amplitude objects may be defined as objects, which lower the amplitude of the light, which is transmitted through the amplitude object. According to this definition, non-transparent objects are amplitude objects.

The microscope system may be configured as a bright-field microscope system. The microscope system may include an illumination system, which is configured to illuminate the object by transmitting light through the object.

The microscope system may be configured to detect the image, which is generated in the image plane. The image plane may be defined by a sensitive surface of an image sensor and/or defined by a focal plane of an eyepiece. The image plane and the object plane are optically conjugate planes. Hence, the object plane may be defined depending on the image plane. Optically conjugate planes may be defined such that a ray bundle, which is emitted from a point in the first plane, is focused on a point in the second plane. The focus plane of an amplitude object, which is located in the object plane, is therefore located in the image plane. However, the focus plane of a phase object, which is located in the object plane, may be located outside the image plane. The phase object may have a plurality of focus planes, which are located in front of and/or behind the image plane, as seen along the beam path.

The focus plane of an object may be defined as a plane, located at an axial position along the optical axis, at which the imaged object has a maximum contrast or a local maximum contrast, compared to other axial positions. In other words, the contrast may decrease when a deviation from the axial position of the focus plane occurs. The focus plane may be orientated perpendicular to the optical axis.

The focus shift data may represent or include a distance between a focus plane of the phase object and a focus plane of the amplitude object, when the phase object and the amplitude object are located in a common plane, which extends perpendicular to the optical axis. The distance may be measured along the optical axis. Alternatively or additionally, the focus shift data may include and/or represent a distance between the object plane and the phase object, wherein the distance between the object plane and the phase object is configured so that a focus plane of the phase object is located in the image plane.

The microscope system may be a monoscopic microscope system or a stereoscopic microscope system. The stereoscopic microscope system may be configured to generate two observation beam paths. The axes of the two observation beam paths may form a stereo angle in the object plane. The stereoscopic microscope system may be configured to generate images in two image planes. The images of the two image planes may represent a stereoscopic pair of images.

The microscope system may include an image sensor, which is arranged in the image plane or in a plane, which is optically conjugate to the image plane. Alternatively or additionally, the microscope system may include an eyepiece, which is configured so that the image in the image plane is imageable onto the retina of an observer.

The microscope system may include a computing system and/or a electronic data storage system. The electronic data storage system may be configured to store the focus shift data. The microscope system may further comprise a controller. The controller may be configured to read the stored focus shift data and to adjust one or more settings of the microscope system depending on the focus shift data. The settings of the microscope system may be one or a combination of a distance of the image plane from the object, a distance between the object plane and the image plane, and a variable optical power of an optical component of the microscope system.

According to an embodiment, the microscope system and/or the controller is configured to adjust, depending on the focus shift data, a distance between the object plane and the object so that a focus plane of the object is located in the image plane. The distance may be measured along the optical axis.

If a focus plane of the phase object is located in the image plane, the phase object can be imaged by the microscope system with high contrast. As a result of the focus shift, the phase object has to be located outside the object plane.

According to an embodiment, the microscope system may be configured to adjust the distance between the object plane and the phase object by adjusting a distance between the image plane and the phase object, as measured along the optical axis of the imaging optics. Thereby, a position of the focus plane relative to the image plane may be adjusted. The microscope system may be configured so that the distance of the image plane from the object is variable. For example, the microscope system may include actuators, which are in signal communication with a controller. The actuators may be drivingly connected to a microscope housing, to an image sensor, to an eye piece and/or to an object holder of the microscope system, so that by actuating the actuator, the distance between the image plane and the object is adjusted. The object holder may, for example, be a head support, on which the head can be placed so that a region of the eye can be imaged by the microscope system.

Additionally or alternatively, the microscope system may be configured to adjust the distance between the object plane and the phase object by adjusting a distance between the image plane and the object plane, as measured along the optical axis of the imaging optics. By way of example, the microscope system may be configured to adjust an axial position of an optical component along the optical axis. The optical component may for example be an objective lens of the imaging optics. The objective lens may have a focal length of more than 75 millimeters, more than 100 millimeters, more than 150 millimeters, more than 200 millimeters, or more than 250 millimeters. The microscope system may include an actuator, which is signal communication with a controller. The actuator may be drivingly connected to the optical component and/or to the image sensor so that actuating the actuator causes the axial position of the optical component and/or the image sensor to be changed.

Additionally or alternatively, the microscope system may be configured to adjust the distance between the object plane and the phase object by adjusting a variable optical power of the imaging optics. The microscope system may include an optical component, which has a variable optical power, in particular a variable spherical optical power. The optical component may be in signal communication with the controller so that, depending on signals received from the controller, the optical power of the optical component is adjusted. By way of example, the optical component is a zoom system, a liquid lens and/or an Alvarez lens.

Additionally or alternatively, the microscope system may have two image planes. The microscope system may be configured to detect each of the images generated in the image planes. Each of the image planes may be optically conjugate to an object plane so that the object planes are spaced apart from each other along the optical axis. The distance may be configured so that the focus shift is compensated by the distance between the object planes. Thereby, it is possible that an amplitude object and a phase object, which are located in a common plane, which is oriented perpendicular to the optical axis, are imaged in focus. The focus plane of the phase object may be located in the first image plane and the focus plane of the amplitude object may be located in the second image plane. The microscope system may be configured such that the distance between the object planes is adjustable depending on signals received from the controller.

According to an embodiment, the adjusting the distance between the object plane and the phase object is further performed depending on an object distance of a reference object. This allows inspection of a region surrounding the reference object in order to determine whether further phase objects are located within the surrounding region.

The object distance may be defined as a distance of the object from the imaging optics, in particular from stationary components of the imaging optics, measured along the optical axis. By way of example, the reference object is an instrument configured manipulate the phase object and/or to interact with the phase object. By way of example, the instrument include capsulorhexis forceps for accomplishing capsulorhexis in the capsular bag. Furthermore, by way of example, the instrument is a cannula. The cannula may be configured for aspiration of residues. The residues may be residues of the natural lens of the eye.

According to a further embodiment, the microscope system is configured to detect an object distance of the reference object. By way of example the microscope system may include position sensors, which are configured so that the object distance of the reference object can be detected.

According to a further embodiment, the microscope system is configured to generate reference object image data, which represent an image of a reference object. The reference object may be an amplitude object. The microscope system may further be configured to adjust the distance between the object plane and the phase object, depending on the reference object image data.

The reference object image data may include a plurality of images. The images may be acquired at different distances of the object plane relative to the reference object. The images may include an in-focus image of the reference object. An in-focus image of an object may be defined as an image, for which the focus plane of the object is located in the image plane. The microscope system may be configured so that the in-focus image is determined from among the plurality of images of the reference object image data. By way of example, the microscope system may compare the image data of the images to determine the image, which has the greatest image sharpness.

The microscope system may be configured to determine, depending on the reference object image data, an object distance of the reference object. The object distance may correspond to a position of the object plane, when the reference object is imaged onto the image plane in-focus.

The microscope system may be configured to position, depending on the determined object distance of the reference object and further depending on the focus shift data, the object plane relative to the eye or relative to the reference object so that objects, which can be imaged as phase objects, and which have a same object distance as the reference object, are imaged in-focus onto the image plane. In other words, the focus plane of the phase object is located in the image plane.

According to a further embodiment, the microscope system is configured to acquire data, which represent a position of the object plane, at which a sign of an imaging intensity of the phase object in the image plane, is reversed, relative to a background intensity in the image plane.

Through the determination of the position of the object plane, at which the sign of the imaging intensity is reversed, the object distance of the phase object can be determined. The position of the object plane may be an axial position relative to the optical axis.

The imaging intensity of the object may be an intensity at a position in the image plane, which corresponds to a position of the object. The background intensity may be an intensity at a position in the image plane, which is outside of any imaged object.

According to a further embodiment, the microscope system is configured to determine an object distance of the phase object, depending on the focus shift data.

According to a further embodiment, the microscope system is configured to generate phase object image data, which represent an image of a further object, which is a phase object. The microscope system may further be configured to determine the focus shift data, depending on the generated phase object image data.

The phase object image data may include a plurality of images, which have been acquired at different distances of the object plane relative to the further phase object. The microscope system may be configured to determine one or more in-focus images from among the plurality of images. The plurality of in-focus images may represent images of the further phase object in different focus planes. The microscope system may be configured to determine the focus shift data depending on the object plane distances of the in-focus images. The object plane distance may be a distance of the object plane from the imaging optics, in particular from stationary components of the imaging optics, measured along the optical axis. By way of example, the focus shift data may be determined depending on two or more object plane distances of in-focus images. In particular, the focus shift data may be determined depending on a distance between two or more of the object plane distances. By way of example, the focus shift data may be determined as one-half of a distance between two object plane distances of in-focus images, which correspond to two neighboring focus planes.

Alternatively or additionally, the focus shift data are determined depending on a known object distance of the further phase object. The object distance may be a distance of the object from the imaging optics, measured along the optical axis.

Alternatively or additionally, the phase object image data may further represent one or more images of an amplitude object, wherein the further phase object and the amplitude object are located in a common plane, which is oriented perpendicular to the optical axis.

According to a further embodiment, the microscope system is configured to determine the focus shift data depending on a spatial frequency to be imaged of a phase object. The spatial frequency may be a spatial frequency of the phase object measured in the object plane. The term frequency to be imaged may be defined to mean that the frequency is resolved by the image of the phase object. The spatial frequency may be a pre-selected frequency. The pre-selected frequency may be selected depending on the objects, which are to be inspected.

According to a further embodiment, the microscope system comprises an illumination system configured to generate a light emitting surface within the eye. The microscope system may be configured to determine the focus shift data depending on a size of the light emitting surface.

The light emitting surface may be an illumination spot on the retina or a light exit surface of a light guide, wherein the light exit surface is disposed in the interior of the eye.

According to a further embodiment, the microscope system further comprises an illumination system, which is configured to generate a red reflex illumination for one or more values of an optical power of the eye.

The illumination system may be configured to generate a red reflex illumination for one or more values of an optical power of the eye. The red reflex is also commonly referred to as red pupillary reflex or red retinal reflex. The optical power may include a spherical and/or a cylindrical optical power. The one or more values of the optical power may be so that the eye is emmetropic, aphakic or has a spherical ametropia in a range of between −20 diopters and +20 diopters.

It has been shown that by using red reflex illumination, the focus shift data can be determined with comparatively high accuracy. Furthermore, the focus shift data then can be used for imaging phase objects in a particularly efficient way.

The illumination system may be configured so that an axis of the illumination beam path, which is incident on the eye, is closely aligned with an axis of the observation beam path. It has shown that thereby, an efficient red reflex illumination can be provided. The range within which the axis of the illumination beam path is in close alignment with the axis of the observation beam path may be defined as a range of an angle, formed between the axis of the illumination beam path and the axis of the observation beam path, of less than 6°, or less than 4°, or less than 2° or less than 1°. In particular, at the object, the axis of the illumination beam path may be coaxial to the axis of the observation beam path.

The illumination system may be configured so that an angle of beam convergence or an angle of beam divergence of the illumination beam path is adjustable at a position, where the illumination beam path enters the eye. By adjusting the angle of beam convergence or angle of beam divergence, it is possible to generate a small-sized illumination spot on the retina for a range of optical powers of the eye. Thereby, it is possible to provide red reflex illumination for aphakic eyes or for eyes having a spherical ametropia.

Alternatively or additionally, the illumination system may include a light guide, such as an optical fiber, which is configured so that a distal end portion of the light guide is insertable into the eye. For example, the end portion may be inserted into the eye through the sclera and brought into a position close to the retina. The light guide may be configured so that light, which is emitted from an end of the optical fiber, illuminates an anterior portion of the eye thereby providing transmitted-light illumination. The light guide may be a single-mode light guide. The light guide may include one or more optical fibers. The light guide may be disposed within a tube. The tube may have a bend so that light, which is emitted from the end of the light guide is directed toward the anterior portion of the eye.

According to an embodiment, the microscope system includes an illumination system configured to generate an illumination beam path. The illumination beam path may have a minimum cross-section from which the illumination beam path diverges. The illumination system may be configured so that at a position of the minimum cross-section the following relation holds:

D·sin(β)<M.

D may denote a diameter of the minimum cross-section of the illumination beam path. 13 may denote an object side angle of divergence of the illumination beam path at the position of the minimum cross-section. M may have a value of 0.9 mm.

Thereby, it is possible to generate an illumination spot on the retina having a small diameter. By way of example, the diameter of the illumination spot on the retina may be smaller than 1 mm, or smaller than 0.1 mm, or smaller than 10 micrometers, or smaller than 5 micrometers.

It has been shown that thereby, the determination and/or use of the focus shift data can be particularly effective.

The minimum cross-section may be a minimum cross-sectional area of the illumination beam path. The illumination beam path may be formed by all ray paths forming the illumination light. The minimum cross-section may be formed by all ray bundles, which traverse the illumination system and reach the retina. The ray bundles may be emitted from a light exit surface. The light exit surface may be formed by a light guide or by a light source, such as a laser light source. Alternatively, the minimum cross-section may be located at a focus position of the illumination beam path or in an aperture of an aperture stop. Alternatively, the minimum cross-section may be located at a beam waist of a laser beam, or may be a cross-section of the laser beam when leaving the laser. The minimum cross-section may be located within an imaging optical system and/or may be located at an interface between a non-imaging optical system and an imaging optical system. A non-imaging optical system may be a light guide such as an optical fiber.

The minimum cross-section may be located in the illumination beam path between the light source and the object plane, in particular between the light source and the objective lens. The light source may include a laser, a halogen lamp, and/or a xenon lamp. The laser may be a gas laser, a solid state laser or a diode laser.

According to an embodiment, the minimum cross-section and the angle of divergence are determined from the light rays, which are emitted from the light source and are incident within a circular region of the object plane, which has a diameter or 6 or 8 mm around the axis of the illumination beam path.

According to a further embodiment, the illumination system is configured such that more than 50%, and in particular more than 80% of a spectral intensity distribution of the illumination light, which is guided to the object plane by the illumination system, is within a wavelength range of between 580 nm and 1,400 nm; or within a wavelength range of between 650 nm and 1,400 nm, or within a wavelength range of between 700 nm and 1,400 nm, or within a wavelength range of between 580 nm and 900 nm, wherein the wavelength range is measured in the object plane.

Thereby, the illumination light is within a wavelength range within which the retina has a high reflectivity. Thereby, it is possible to generate a comparatively small illumination spot by the illumination optics over a long expose time, without causing damage to the retina as a result of the light intensity of the illumination light.

The illumination system may include a light guide through which light is guided to a light emitting surface of the light guide. The light guide may transport the light from a light source or an optical coupler to the light emitting surface. The light emitting surface of the light guide may emit the light into an imaging optical system of the illumination system. The illumination optics may be an imaging optical system of the illumination system. According to an alternative embodiment, the light emitting surface of the light guide is disposed within the interior of the eye.

According to a further embodiment, the illumination system includes a single mode light guide. The light guide may be an optical fiber. By using a single mode light guide, a particularly efficient illumination system is provided for generating an illumination beam path having a comparatively small value for D·sin(β).

Embodiments provide a method of operating a microscope system for imaging an object. The object may be imaged by the microscope system as a phase object. The microscope system may comprise imaging optics for generating images of an object plane of the imaging optics in an image plane of the imaging optics by using imaging light. The object plane may be optically conjugate to the image plane. The method may comprise generating focus shift data representing a focus shift, wherein the focus shift is generated by a different effect on the imaging light generated by phase objects as compared to amplitude objects. The method may further comprise imaging the object with the microscope system, depending on the determined focus shift data.

The microscope system may be a bright-field microscope. The microscope may have an illumination system for illuminating the object by transmitted light.

According to an embodiment, the method neither comprises methods for treatment of the human or animal body by surgery or therapy. According to a further embodiment, the method does not include diagnostic methods practiced on the human or animal body.

According to a further embodiment, the method further comprises adjusting a distance between the object plane and the phase object so that a focus plane of the phase object is located in the image plane, depending on the generated focus shift data.

According to a further embodiment, the adjusting the distance between the object plane and the object is performed depending on an object distance of a reference object.

According to a further embodiment the method further comprises generating reference object image data, which represent an image of a reference object, wherein the reference object is an amplitude object. The adjustment of the distance between the object plane and the phase object may be performed depending on the reference object image data.

According to a further embodiment, the method comprises acquiring data, which represent a position of the object plane, at which a sign of an image intensity of the phase object in the image plane, which is measured relative to a background intensity in the image plane, reverses.

According to a further embodiment the method further comprises determining an object distance of the phase object depending on the focus shift data.

According to a further embodiment the method further comprises generating phase object image data, which represent an image of a further object, which is a phase object. The method may further include determining the focus shift data depending on the phase object image data.

According to a further embodiment the method further comprises determining the focus shift data depending on a spatial frequency to be imaged of a phase object.

According to a further embodiment the method further comprises determining the focus shift data depending on a size of a light emitting surface of an illumination system. The light emitting surface may be disposed within the eye.

Embodiments provide a non-transitory computer-readable storage medium storing instructions that, when executed by a computer, cause the computer to perform a method of operating a microscope system for imaging an object. The object may be imaged by the microscope system as a phase object. The microscope system may comprise imaging optics for generating images of an object plane of the imaging optics in an image plane of the imaging optics by using imaging light. The object plane may be optically conjugate to the image plane. The method may comprise generating focus shift data representing a focus shift, wherein the focus shift is generated by a different effect on the imaging light generated by phase objects as compared to amplitude objects. The method may further comprise imaging the object with the microscope system, depending on the determined focus shift data.

According to a further embodiment, the phase object is a lens residue, in particular a piece of lens residue in an eye. The lens residue may be generated in the process of removing the natural lens, for example during cataract surgery. The reference object may be an instrument for picking up and/or aspirating the lens residue. The method may comprise determining the lens residue in a capture region and/or a working region of the instrument. The capture region and/or working region may be a spatial region in the interior of the eye, from which lens residues can be picked up while maintaining the instrument at a constant position and/or orientation. The instrument may be a hollow cannula and/or a pair of tweezers.

### BRIEF DESCRIPTION OF THE DRAWINGS

The forgoing as well as other advantageous features of the disclosure will be more apparent from the following detailed description of exemplary embodiments with reference to the accompanying drawings. It is noted that not all possible embodiments necessarily exhibit each and every, or any, of the advantages identified herein.

FIGS. 1A, 1B and 1C schematically illustrate stages during cataract surgery;

FIG. 2 schematically illustrates a microscope system according to an exemplary embodiment;

FIG. 2B schematically illustrates an illumination system of a microscope system according to an alternative exemplary embodiment;

FIG. 3 schematically illustrates a focus shift, which is generated by different effects of phase objects on the imaging light, as compared to amplitude objects;

FIGS. 4A, 4B and 4C schematically illustrate the imaging of phase objects depending on focus shift data;

FIG. 5 schematically illustrates the influence of the size of the illumination spot on the coherence of the imaging light;

FIGS. 6A and 6B schematically illustrate the minimum cross-section of the illumination beam path according to an exemplary embodiment;

FIGS. 7A and 7B illustrate the observation light path of a microscope system according to an exemplary embodiment.

### DETAILED DESCRIPTION OF EXEMPLARY EMBODIMENTS

It should be noted in this context that the terms “comprise”, “include”, “having” and “with”, as well as grammatical modifications thereof used in this specification or in the claims, indicate the presence of technical features such as stated components, figures, integers, steps or the like, and by no means preclude the presence or addition of one or more alternative features, particularly other components, figures, integers, steps or groups thereof.

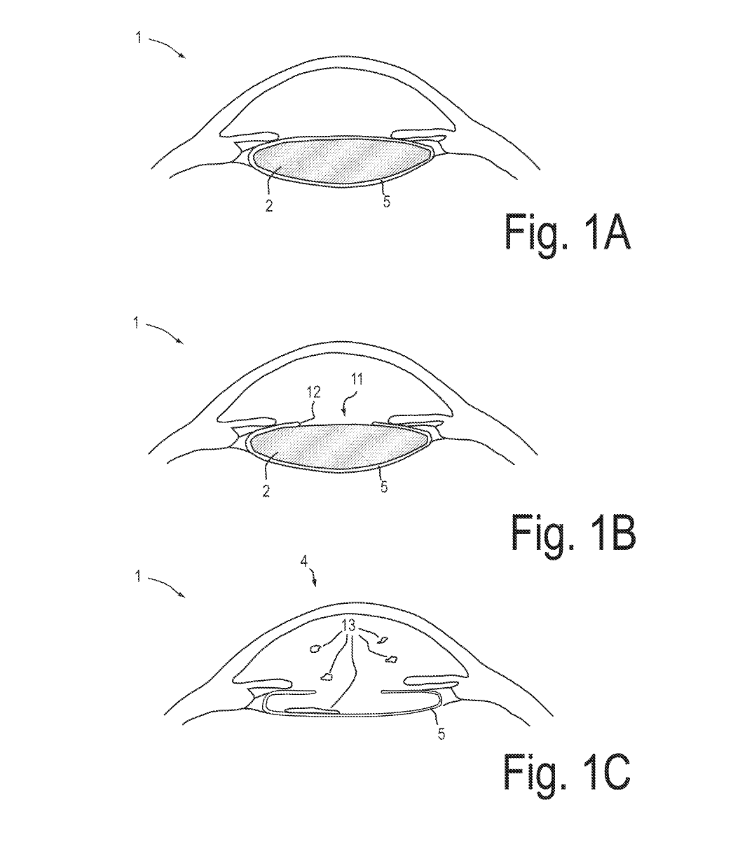

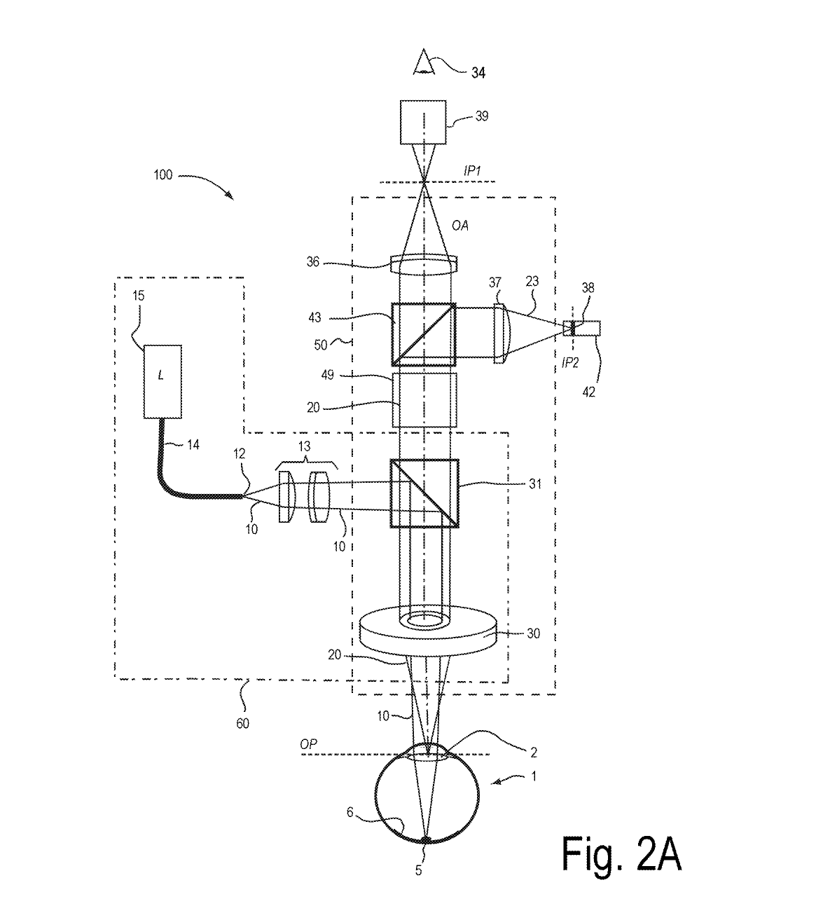

FIGS. 1A, 1B and 1C illustrate stages during cataract surgery, performed on an eye 1. In cataract surgery, the opaque natural lens 2 is removed and replaced by an intraocular lens (not shown). FIG. 1A shows the eye before the surgery is initiated. The opaque natural lens 2 is surrounded by a capsular bag 5. In capsulorhexis, which is a commonly performed surgical procedure, an opening 11 (shown in FIG. 1B) is generated in an anterior portion of the capsular bag 5. The thereby created edge of the opening 11 (also called edge of the rhexis), which surrounds the opening 11, should be smooth and continuous to prevent lacerations in the capsular bag and to allow perfect positioning of the intraocular lens. After finishing the capsulorhexis, the natural lens is usually shattered by ultrasound (phakoemulsification). Lens residues 13 (shown in FIG. 1C), which are thus created, are usually aspirated through a hollow cannula. If not all of the lens residues 13 are removed from the eye, this can cause posterior capsule opacification.

The edge of the rhexis and the lens residues 13 are—like many other tissue portions of the eye—substantially phase objects. When light passing through the phase object, the amplitude of the light remains substantially unchanged but the phase is changed. Phase objects are distinguished from amplitude objects. Amplitude objects significantly decrease the amplitude of the transmitted light.

The following exemplary embodiments allow efficient detection and manipulation of phase objects.

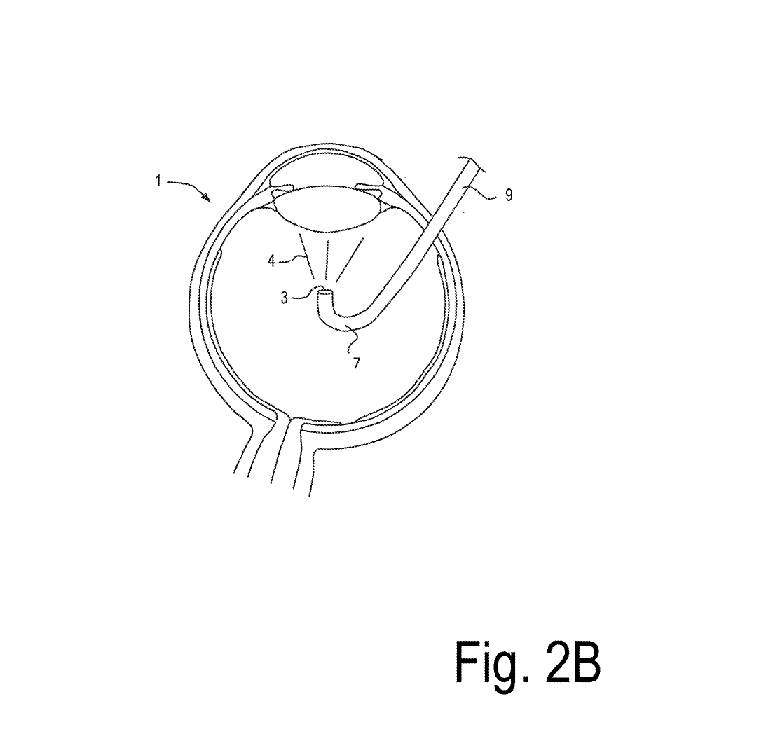

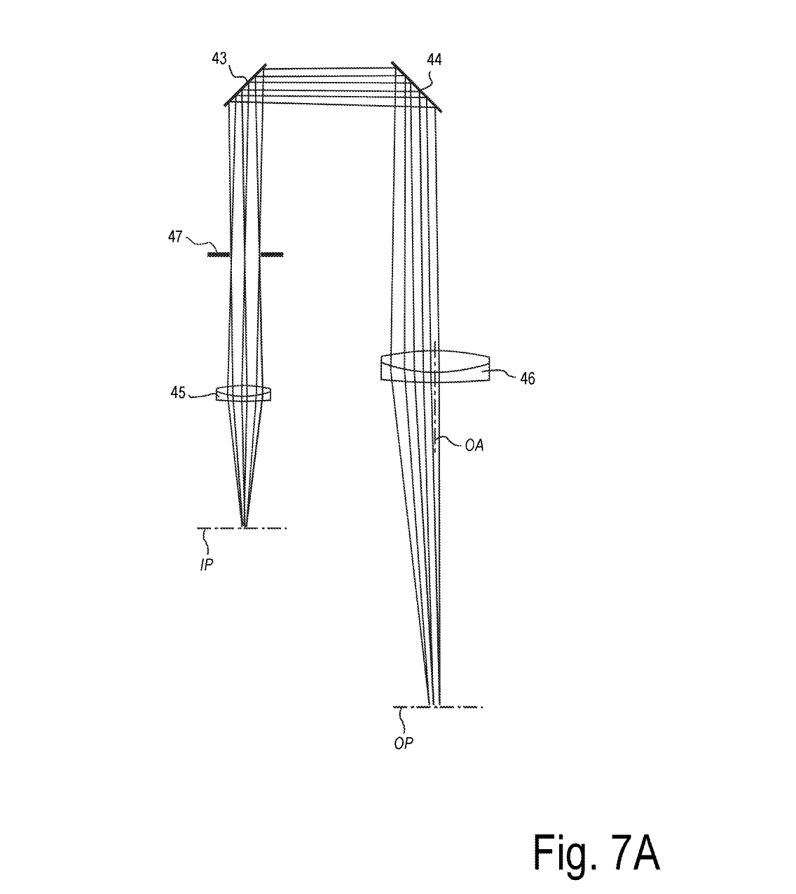

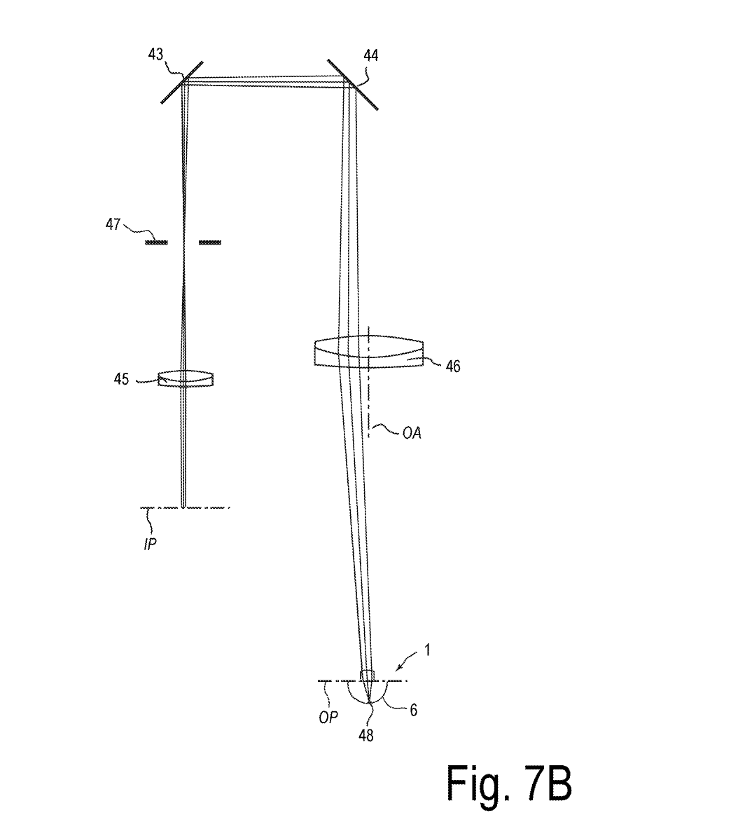

FIG. 2A shows a microscope system 100 according to a first exemplary embodiment, which is configured to image a portion of an eye and which allows more efficient imaging of phase objects. The microscope system 100 is configured as a monoscopic microscope. As is described with reference to FIGS. 7A and 7B, it is also possible that the microscope system is configured as a stereoscopic microscope.

The microscope system 100 includes an illumination system 60 and imaging optics 50. The illumination system 60 includes a laser 15 and a light guide 14, which is an optical fiber configured as a single mode optical fiber. Through the light guide 14, light of the laser 15 is guided to illumination optics, configured as an imaging optical system. The illumination optics include optics 13, a beam splitter 31 and the objective lens 30. The beam splitter 31 and the objective lens 30 are traversed by the observation beam path 20. Thereby, the beam splitter 31 and the objective lens 30 form part of the illumination system 60, as well as part of the imaging optics 50.

The illumination system 60 is configured such that in a region of the object plane OP, an axis of the illumination beam path 10 is closely aligned with an axis of the observation beam path 20. In other words, at the object, the axis of the illumination beam path 10 and the axis of the imaging light path 20 form an angle which is less than 6°, or less than 4°, or less than 2°, or less than 1°. This configuration allows red reflex illumination of the eye 1.

Furthermore, the illumination system 60 is configured such that the incident light is focused onto the retina 6 of the eye 1 under inspection. In the exemplary embodiment, which is shown in FIG. 2A, a light exit surface 12 of the light guide 14 is imaged onto the retina 6 of the eye 1. Thereby an illumination spot 5 is generated on the retina 6. At the illumination spot 5, the incident light is diffusedly scattered such that reflected light is emitted from the illumination spot 5 having spherical or substantially spherical wave fronts. Thereby, the anterior portion of the eye 1 is illuminated by transmitted light.

Imaging optics 50 include a further beam splitter 43 for extracting light from the imaging light path. The extracted portion of light is guided to image plane IP2 via a lens 37. The non-extracted portion of the light is guided to image plane IP1 via a lens 36. Each of the image planes IP1 and IP2 is optically conjugate to the object plane OP.

An image sensor 38 of a camera 42 is disposed in the image plane IP2. The focal plane of an eyepiece 39 is located in the image plane IP1. Thereby, image plane IP2 can be imaged by eyepiece 39 onto the retina of a user 34.

Furthermore, the imaging optics 50 includes a zoom system 49 which may, for example, be configured as an afocal zoom system.

FIG. 2B illustrates an illumination system of a microscope system according to an alternative exemplary embodiment. The imaging optics of the microscope system according to this alternative exemplary embodiment is of the same design as in the exemplary embodiment which is shown in FIG. 2A. The illumination system of the alternative embodiment includes a light guide, the end portion of which is insertable into the interior of the eye 1. In the alternative exemplary embodiment, which is shown in FIG. 2B the end portion of the light guide is disposed within a tube 9. The tube 9 includes a curved section 7 so that light from a light exit surface 3 of the light guide illuminates an anterior section of the eye thereby forming transmitted-light illumination of the anterior section.

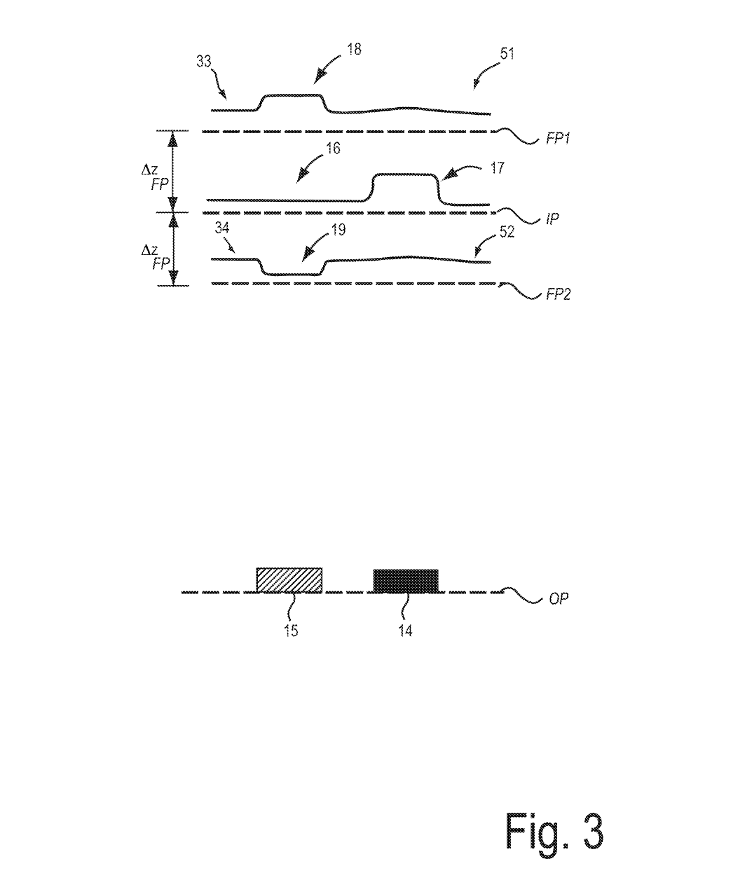

The microscope system is configured to store focus shift data and to perform the imaging of phase objects depending on the stored focus shift data. The focus shift data represent a focus shift, which is generated by different effects of amplitude objects and phase objects on the imaging light. The focus shift is schematically illustrated in FIG. 3.

FIG. 3 shows and amplitude object 14 and a phase object 15, each of which being disposed in the object plane OP of the microscope system. The object plane OP is defined as a plane, which is optically conjugate to an image plane IP of the microscope system. The image plane IP is defined as a plane, in which an image is acquirable by the microscope system. By way of example, in the image plane IP an image sensor of the microscope system or a focal plane of an eyepiece is arranged.

The amplitude object 14 is imaged by the imaging optics onto the image plane IP of the microscope system to generate an in-focus image. This is schematically illustrated by the intensity distribution 17 in FIG. 3. Outside of the image plane IP, the amplitude object 14 is imaged out of focus. This is schematically illustrated by intensity distributions 51 and 52. Hence, the image plane IP is the focus plane of the amplitude object 14.

Phase object 15 is imaged by the imaging optics onto one of more focus planes FP1, FP2, each of which being located outside of the image plane IP, as seen along the imaging beam path. In the image plane IP, at a position 16, which corresponds to the position of the phase object 15, there is no intensity or only a comparatively small intensity detectable. Focus plane FP2 is located, as seen in a direction of the imaging beam path, upstream of the image plane IP, whereas focus plane FP1 is located downstream of the image plane IP. The focus planes FP1 and FP2 are located at a distance Δz_{FP} from the image plane. Thereby, the value Δz_{FP} represents focus shift data representing a focus shift. The focus shift is generated by different effects on the imaging light, caused by amplitude objects and phase objects.

Depending on the properties of the imaging optics, the illumination system and the object, it is conceivable that the phase object 15 is only imaged onto one focus plane or onto more than two focus planes. A prerequisite for the existence of more than two focus planes is, in particular, a high spatial coherence of the imaging light, which is transmitted through the phase object.

Depending on properties of the imaging optics, the illumination system and the object, it is conceivable that the phase object 15 is imaged onto a single focus plane or imaged onto more than two image planes. A prerequisite for imaging the phase object onto more than two focus planes may be a high spatial coherence of the light, which is transmitted through the phase object.

An imaging intensity 18 of the phase object in the focus plane FP1, which is measured relative to a background intensity 33 in the focus plane FP1, has an opposite sign relative to an imagining intensity 19 in the focus plane FP2, which is measured relative to a background intensity 34 in the focus plane FP2. The image plane IP is the plane in which the sign of the imaging intensity is reversed. This allows distinguishing phase objects and amplitude objects by varying the position of the object plane OP along the optical axis. Furthermore, by determining the position of the object plane OP, at which the sign reverses, the object distance of the phase object 15 can be determined.

The microscope system is configured to store the value Δz_{FP}. As is described below, the microscope system may further be configured to generate the focus shift data.

The microscope system is configured to vary the position of the object plane OP relative to the phase object 15 so that the phase object 15 is located outside the object plane OP. The microscope system is configured to adjust a distance between the object plane OP and the phase object 15 so that one of the focus planes FP1, FP2 is shifted into the image plane IP, in which the image sensor and/or the focal plane of the eyepiece is located. In this configuration, it is possible to image the phase object 15, whereby, however, the amplitude object 14 is not imaged or only imaged out of focus.

The value of Δz_{FP} may be, for example, within a range of between 0.1 millimeters and 2 millimeters.

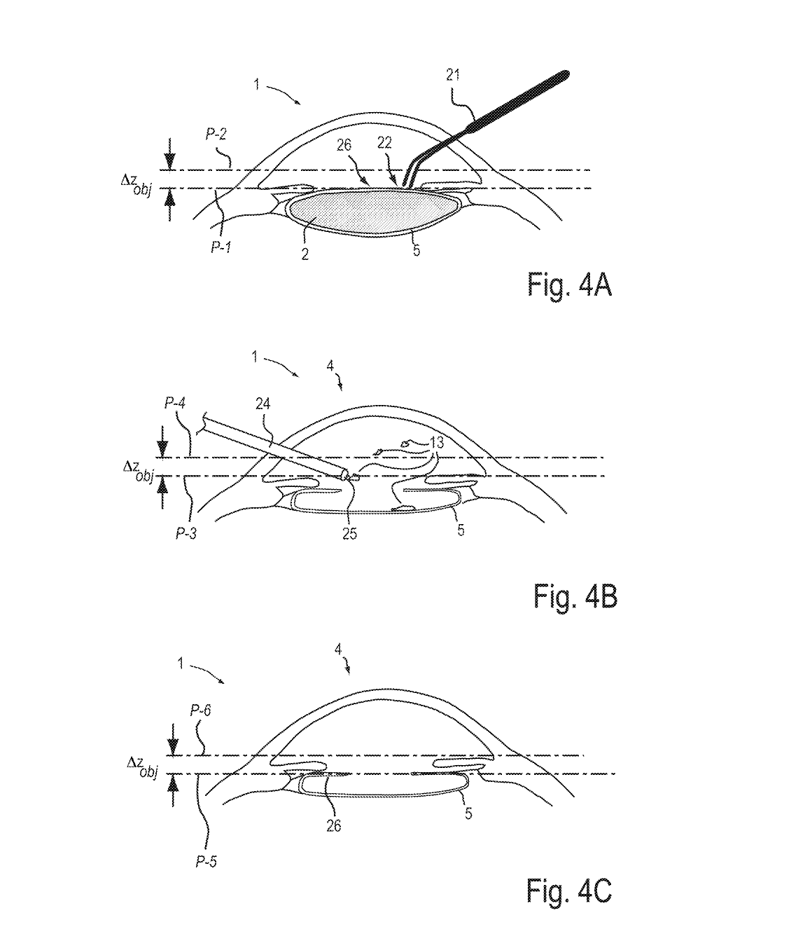

As is illustrated in FIGS. 4A, 4B and 4C, this configuration of the microscope system allows efficient imaging of phase objects, for example, when objects within the eye are to be imaged. In particular, it is possible to comparatively quickly acquire images of phase objects and amplitude objects, compared to conventional techniques of dark-field microscopy or phase contrast microscopy.

FIG. 4A illustrates the performance of capsulorhexis, in which an opening is formed in the anterior portion 26 of the capsular bag 5 by using capsulorhexis forceps 21. In this stage of the procedure, an anterior portion of the capsular bag 5 is gripped by a distal end 22 of the forceps 21. The distal end 22 of the forceps 21 is an amplitude object, whereas the anterior portion 26 of the capsular bag is a phase object. In this situation, focused images of both objects can quickly be acquired by using focus shift data.

For generating a first image the object plane of the microscope system is positioned in the plane P-1. Thereby, an in-focus image of the distal end 22 of the forceps 21 can be acquired in the image plane IP (shown in FIG. 3) of the microscope system. The anterior portion 26 of the capsular bag 5 and the distal end 22 of the capsulorhexis forceps 21 are located in a common plane. Thereby, in the first image, the anterior portion 26 of the capsular bag 5 is only scarcely perceptible or not perceptible at all.

For acquiring a second image, the microscope system is configured to shift the object plane along the optical axis so that an in-focus image of the anterior portion 26 of the capsular bag 5, which is a phase object, can be acquired in the image plane. By way of example, the microscope system may shift the object plane from plane P-1 to plane P-2. The plane P-2 is located displaced relative to the plane P1 toward the objective lens by an amount of Δz_{OP}.

Alternatively, the microscope system may be configured to shift the object plane from plane P1 to a plane, which has a distance from the imaging optics, which is greater than the distance of plane P1 from the imaging optics, by an amount of Δz_{OP}.

Thereby, one of the focus planes FP1, FP2 (shown in FIG. 3) of the anterior portion 26 of the capsular bag 5 is positioned in the image plane.

Alternatively, the microscope system may shift the object plane in a direction toward the imaging optics or in a direction away from the imaging optics by an amount, which is an integral multiple of Δz_{OP}.

The amount Δz_{OP}, by which the object plane needs to be displaced in order to position one of the focus planes FP1 or FP2 of a phase object, which is initially located in the object plane, into the image plane is

$$Δ {z}_{OP} = \frac{Δ {z}_{FP}}{T} , \qquad ( 1 )$$

wherein Δz_{FP} (shown in FIG. 3) is the focus shift of one of the focus planes FP1, FP2 of the phase object 15, measured relative to the focus plane of the amplitude object 14. The value of Δz_{FP} therefore represents focus shift data. The parameter T denotes the magnification along the depth direction, for which the following relation holds:

$$T = \frac{{M}^{2}}{\hat{η}} , \qquad ( 2 )$$

wherein M is the imaging magnification for imaging the object plane onto the image plane. The parameter \hat{η} is defined by the expression:

$$\hat{η} = \frac{{n}_{obj}}{{n}_{im}} , \qquad ( 3 )$$

wherein n_{obj} is the refractive index of the medium within which the object plane is located and nᵢₘ is the reflective index of the medium within which the image plane is located. The medium within which the image plane is located may be air, having a refractive index of nᵢₘ=1. The medium within which the object plane is located, may be the aqueous humor of the eye, having a refractive index of n_{obj}=1.33.

Thereby it is possible to generate an in-focus image of the distal end 22 of the forceps 21, as well as of the portion of the anterior portion 26 of the capsular bag 5, which has been manipulated by using the capsulorhexis forceps 21. The microscope system may be configured to generate a further image depending on the first and second images so that the user can simultaneously perceive in-focus images of the distal portion 22 of the forceps 21 and the anterior portion 26 of the capsular bag 5 in a single image.

FIG. 4B shows a cannula 24, which has been inserted into the interior of the eye 1 for removing lens residues 13, which have been generated by shattering the natural lens. The microscope system includes position sensors (not shown). A controller of the microscope system is configured to measure a position along the optical axis and/or an orientation of the entry opening 25 of the cannula 24 by using the position sensors. Thereby, an object distance of the entry opening 25 from the imaging optics can be determined.

The entry opening 25 of the cannula 24 is located in the plane P-3. The microscope system is configured to position the object plane in the plane P-4, depending on the focus shift data. The plane P4 is shifted relative to the plane P-3 in a direction along the optical axis toward the imaging optics by an amount of Δz_{obj}. Alternatively, the microscope system may, shift the object plane into a plane, which has a distance from the imaging optics, which is greater than the distance of the plane P-3 from the objective lens by an amount of Δz_{obj}.

Thereby, it is possible to generate an in-focus image in the image plane from lens residues, which are phase objects and which are located in the vicinity of the entry opening 25 of the cannula 24.

FIG. 4C illustrates a state of the eye 1 after the lens residues 13 (shown in FIG. 4B) have been removed using the cannula 24, shown in FIG. 4B. The microscope system is configured to move the object plane along the optical axis of the microscope system toward the retina, starting from a region of the cornea 4, until an in-focus image of the anterior portion 26 of the empty capsular bag 5 is generated in the image plane. The anterior portion 26 is located in the plane P-5. Hence, the object plane is located in the plane P-6, which is located shifted relative to the plane P-5 by an amount of Δz_{obj}.

The microscope system is configured so that the position of the object plane relative to the eye 1 is determinable. The position of the object plane may be determined from parameters defining the imaging beam path. Depending on the stored focus shift data and depending on the position of the object plane, it is possible to determine the position of the anterior portion 26 of the capsular bag 5 relative to the eye 1.

Alternatively, the microscope system may be configured to determine both object plane distances, at which the first and the second focus planes FP1, FP2 (shown in FIG. 3) are located in the image plane. The average value of these two objects plane distances represents the object distance of the anterior portion 26 of the capsular bag 5.

The determined position of the anterior portion 26 relative to the eye 1 may be used to select the intraocular lens, which is to be implanted into the eye. In particular, it has been shown that, depending on the position of the anterior portion 26 of the capsular bag 5, the optical power of the intraocular lens, which is to be implanted, can reliably be determined.

For performing the exemplary methods, which are illustrated in FIGS. 4A to 4C, the microscope system is configured to adjust the object plane relative to the eye, depending on the focus shift data.

By way of example, the microscope system is configured so that a distance between the image plane and the object, measured along the optical axis, can be varied. For example, the microscope housing may be movably supported relative to the object. Alternatively or additionally, the microscope system may be configured so that the distance between the image plane and the object plane, measured along the optical axis, can be adjusted. By way of example, the microscope system may be configured such that the axial position of an optical component can be adjusted relative to the optical axis. The optical component may be, for example, an objective lens.

Alternatively or additionally, the microscope system may include an optical component, which has a variable optical power, in particular a variable spherical optical power. The optical component, which has a variable refractive power, may be, for example, a liquid lens.

The microscope system may be configured to generate the focus shift data.

By way of example, the microscope system is configured to generate the focus shift data from image data, which represent images of phase objects. For example, depending on the image data, those positions of the object plane are determined, at which a focus plane of the phase object is located in the image plane. Alternatively, the microscope system may be configured to determine the focus shift data according to the following equations.

For purposes of simplification of the illustration, in the following equations, only one spatial coordinate (“x”) is indicated. However, it is possible to generalize the equations to spatial coordinates “x” and “y”.

Immediately after the light wave has been transmitted through the object, the light wave can be expressed by:

U(x)=U₀(x)·exp(iV(x)), (4)

wherein U₀(x) represents the spatial dependency of the wave function, which is incident on the object. V(x) represents the spatial dependency of the phase shift, introduced into the wave function as the wave is transmitted through the phase object. Since an ideal phase object does not decrease the amplitude of the light, the function V(x) only has real values.

The intensity I(x) in the image plane, which is generated by imaging the object with spatially coherent light, can be expressed by the square of the absolute value of a convolution of the complex point spread function with the function U(x):

I(x)=|U(x)\textcircled{×}H(x)|² (5)

The convolution integral of equation 5 can be simplified by using the weak phase approximation when the following relation holds true:

V(x)<<2·π, (6)

wherein V(x) is the phase shift, which is introduced into the wave, which incident on the object plane, after the wave has been transmitted through the object.

In the weak phase approximation, the expression 1+iV(x) is substituted for the factor exp(iV(x)) into equation (5). Hence, the intensity I(x) in the image plane can be approximately expressed by the following equation:

I(x)=I₀(1+2V(x)\textcircled{×}ImH(x)), (7)

wherein I₀ is the background intensity in the image plane.

The point spread function is in general a complex-valued function. However, the point spread function has real values if the object is imaged without aberrations. From equation 7, it can be seen that in this case the intensity is constant in the image plane. Hence, in the image plane, a phase object can not be detected by recording the intensity of light.

In what follows, the intensity distribution is determined outside of the image plane in order to determine at which positions, measured along the optical axis, the focus planes of the imaged phase object are located. To this effect, based on equation 7, an expression of the intensity in a plane is derived, which is displaced relative to the image plane along the optical axis by a distance Δz.

Given the assumption that the influence of the aberrations on the point spread function is negligible compared to the influence of the diffraction on the point spread function. Then, the point spread function can be approximated by a quadratic term in the wave function on a plane, which is displaced by a distance Δz relative to the image plane. By using Fourier's theorem, equation (7) can be transformed into the following equation, which is an integral over the angular frequency v:

$$I ( x , Δ z ) = {I}_{0} ( 1 - 2 \sqrt{\frac{2}{π}} {\int }_{- NA / λ}^{NA / λ} \frac{\begin{matrix}FT [ V ( x ) ] cos ( v \cdot x / M ) sin \\ ( \frac{π \cdot Δ z \cdot λ \cdot {v}^{2} \cdot \hat{η}}{{M}^{4}} )\end{matrix}}{M} d v ) , \qquad ( 8 )$$

wherein NA denotes the numerical aperture of the system, and A denotes the wavelength of the imaging light. FT[V(x)] is the Fourier transform of the function V(x) and M denotes the imaging magnification for imaging the object plane onto the image plane. The parameter \hat{η} denotes the ratio of the refractive index of the medium of object plane n_{obj} over the refractive index of the medium of the image plane nᵢₘ:

$$\hat{η} = \frac{{n}_{obj}}{{n}_{im}} . \qquad ( 9 )$$

For a given function V(x) and by using equation (8), it is therefore possible to determine the intensity distribution in a plane as a function of a distance Δz of the plane from the image plane.

In what follows, it is assumed that the function V(x) has a cosine characteristic:

V(x)=Δφ·cos(ω·x), (10)

having an amplitude Δφ and a phase object frequency ω. Any phase object can be represented by a series summation of cosine-shaped phase objects of different phase object frequencies.

Inserting expression (10) into equation (8) yields the following expression:

$$I ( x , Δ z ) = {I}_{0} ( 1 + 2 ( \frac{Δφ}{M} ) \cdot cos ( \frac{x \cdot ω}{M} ) \cdot θ ( \frac{Na}{λ} - ω ) sin ( \frac{π \cdot Δ z \cdot λ \cdot {ω}^{2} \cdot \hat{η}}{{M}^{4}} ) ) , \qquad ( 11 )$$

wherein θ denotes the Heaviside function.

From equation (11), it can be seen that the deviation of the intensity I(x,Δz) from the background intensity I₀ is proportional to the amplitude of the phase object (i.e. proportional to Δφ). Furthermore, it can be seen from equation (11) that as a result of the factor θ(Na/λ−ω), the intensity variation has a value of zero for phase object frequencies ω, which are greater than NA/λ since then, the Heaviside function is defined to be zero.

Furthermore, it can be seen from equation (11) that as a result of the factor sin(π·Δz·λ·ω²·\hat{η}/M⁴), the intensity varies depending on the distance Δz from the image plane, wherein at the position of the image plane (i.e. Δz=0) the sign of the intensity reverses and the intensity has a value of zero.

The contrast may be defined as the difference between the brightest point and the darkest point of an image. It follows from equation (11) that for each value of Δz_{FP} the maximum and minimum intensity values can be calculated as follows:

$${I}_{max / min} ( Δ z ) = {I}_{0} ( 1 \pm \frac{2 \cdot Δφ \cdot sin ( \frac{π \cdot Δ z \cdot λ \cdot {ω}^{2} \cdot \hat{η}}{{M}^{4}} )}{M} ) , \qquad ( 12 )$$

wherein it has been assumed that phase fluctuations, which are introduced into the wave by the interaction of the wave with the phase object, can be resolved by the microscope (i.e. θ(NA/λ−ω)=1). Then, the contrast is proportional to the difference between both values:

$$Contrast = {I}_{max} ( Δ z ) - {I}_{min} ( Δ z ) ∝ \vert sin ( \frac{π \cdot Δ z \cdot λ \cdot {ω}^{2} \cdot \hat{η}}{{M}^{4}} ) \vert . \qquad ( 13 )$$

It follows from equation (13) that a maximum contrast occurs at a distance Δz_{FP} from the image expressed by the following equation:

$$Δ {z}_{FP} = \pm \frac{{M}^{4}}{2 \cdot λ \cdot {ω}^{2} \cdot \hat{η}} . \qquad ( 14 )$$

Accordingly, equation (14) defines the position of the focus planes of the phase object, measured relative to the image plane, when the phase object is located in the object plane. Thereby the value Δz_{FP} represents focus shift data.

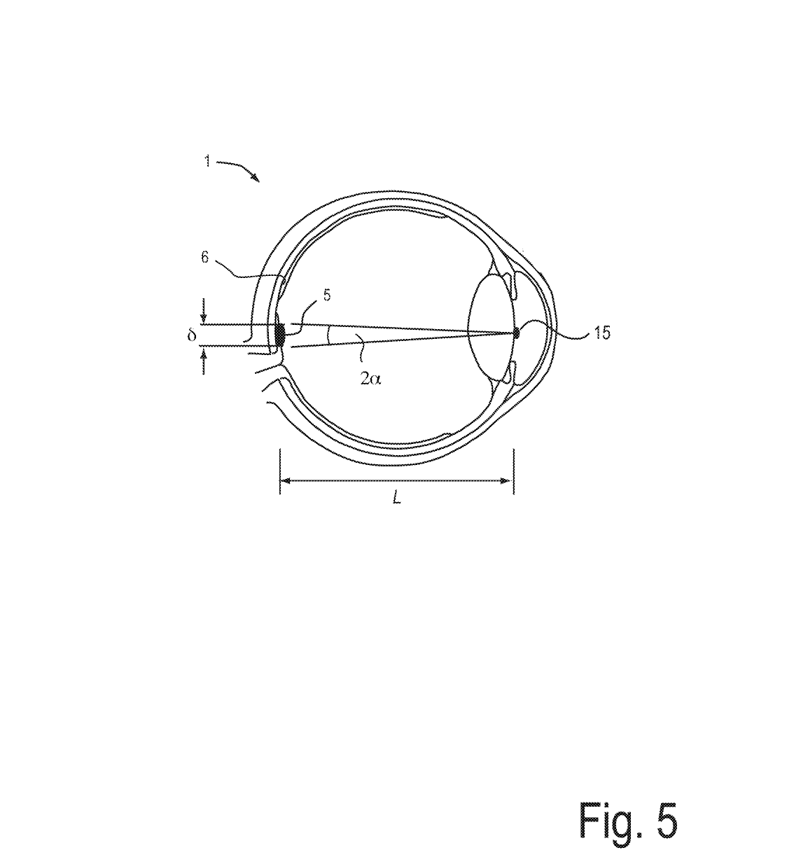

Although, it can be seen from equation (13) that in addition to the positions Δz_{FP}, intensity peaks also occur at further positions. However, as will be discussed in the following with reference to FIG. 5, these further intensity peaks are lowered, in particular as a result of the fact that the light, which is transmitted through the phase object, does not have a high degree of coherence.

If the object plane is shifted relative to the phase object, such that one of the focus planes is located in the image plane, the phase object can be imaged in the image plane. Then, the phase object is located out of the object plane.

The above calculation is based on the assumption that the light, which illuminates the object, has a high degree of coherence. However, it has been shown that in the microscope system, which has been described with reference to FIGS. 2A and 2B, the value for Δz_{FP} may depend on the size of a light emitting surface formed by the illumination spot 5 (shown in FIG. 1) on the retina or by a light exit surface 3 (shown in FIG. 2B) of the light guide. These parameters have an influence on the coherence of the light, which illuminates the object.

As a result of the extent of the illumination spot on the retina or the extent of the light exit surface of the light guide in the interior of the eye, the object is not illuminated with an ideal spherical wave. By considering the size of the illumination spot or the size of the light emitting surface, the illuminating light may be represented by a series summation of plane partial waves, wherein the wave vectors of the partial waves form a cone.

The angle of divergence of this cone is given by the divergence half angle α according to the following equation and the schematic illustration of FIG. 5:

$$α = a tan ( \frac{δ}{2 \cdot L} ) , \qquad ( 15 )$$

wherein δ denotes the diameter of the illumination spot 5 on the retina and L denotes the distance between the illumination spot 5 and the phase object 15, as measured along the optical axis of the microscope system.

The image in the image plane can therefore be expressed as an incoherent sum of partial images, wherein each of the partial images is generated by one of the partial waves. The incoherent sum can lead to a deterioration of the contrast, since the partial waves are not coherently superimposed in the image plane.

By using equations (8) and (15), the following equation can be derived:

$$I ( x , Δ z ) = {I}_{0} \cdot ( 1 - 2 \sqrt{\frac{2}{π}} \cdot {\int }_{- NA \text{/} λ}^{NA \text{/} λ} \frac{\begin{matrix}FT [ V ( x ) ] exp ( - \frac{{π}^{2} \cdot {a}^{2} \cdot Δ {z}^{2} \cdot {v}^{2} \cdot {\hat{η}}^{2}}{{M}^{4}} ) \\ cos ( \frac{v \cdot x}{M} ) sin ( \frac{π \cdot Δ z \cdot λ \cdot {υ}^{2} \cdot \hat{η}}{{M}^{4}} ) \end{matrix}}{M} d υ ) \qquad ( 16 )$$

By comparing equations (16) and 8, it can be seen that the additional factor

$$exp ( - \frac{{π}^{2} \cdot {a}^{2} \cdot Δ {z}^{2} \cdot {v}^{2} \cdot {\hat{η}}^{2}}{{M}^{4}} ) \qquad ( 17 )$$

of equation 16 acts as a low pass filter.

By integrating equation (16) for a phase object according to equation (10), the following intensity distribution is obtained:

$$I ( x , Δ z ) = {I}_{0} \cdot ( 1 + 2 exp ( - \frac{{π}^{2} \cdot {a}^{2} \cdot Δ {z}^{2} \cdot {ω}^{2} \cdot {\hat{η}}^{2}}{{M}^{4}} ) \cdot Δφ \cdot cos ( \frac{x \cdot ω}{M} ) \cdot θ ( \frac{Na}{λ} - ω ) sin ( \frac{π \cdot Δ z \cdot λ \cdot {ω}^{2} \cdot \hat{η}}{{M}^{4}} ) ) , \qquad ( 18 )$$

which yields the following equation for the contrast:

$$Contrast = {I}_{max} ( Δ z ) - {I}_{min} ( Δ z ) ∝ \vert exp ( - \frac{{π}^{2} \cdot {a}^{2} \cdot Δ {z}^{2} \cdot {ω}^{2} \cdot {\hat{η}}^{2}}{{M}^{4}} ) \cdot Δφ \cdot sin ( \frac{π \cdot Δ z \cdot λ \cdot {ω}^{2} \cdot \hat{η}}{{M}^{4}} ) \vert \qquad ( 19 )$$

From equation (19), it is possible to derive the extent to which the positions of the focus planes of the phase object are affected by the size of the illumination spot or the size of the light emitting surface (represented by parameter α, as described with reference to FIG. 5).

For small values of the half angle of divergence α, i.e.

$$α < 0 , 6 \cdot \frac{λ \cdot ω}{π \cdot {M}^{2}} , \qquad ( 20 )$$

the distance Δz_{FP} between the focus plane (i.e. the plane having the greatest contrast) and the image plane (i.e. the focus plane of amplitude objects, which are located in the object plane) does not depend on α. Hence, for these configurations of the illumination system, equation (14) holds true.

For large values of the half angles of a divergence α, i.e.

$$α > 3 \cdot \frac{λ \cdot ω}{π \cdot {M}^{2}} \qquad ( 21 )$$

the distances of the focus planes from the image plane are obtained from equations (14) and (19):

$$Δ {z}_{FP} = \pm \frac{{M}^{2}}{\sqrt{2} π \cdot α \cdot \hat{η} \cdot ω} . \qquad ( 22 )$$

It follows from equation (22) that the distance between the focus plane and the image plane decreases with increasing half angle of divergence.

Also important for practical applications are configurations which have a medium-sized half angle of divergence α, for which neither inequality (20) nor inequality (21) holds true. From equation 19, it can be shown that for these configurations, the distances of the focus planes correspond to the maximum values of the following function:

$$exp ( \frac{- {π}^{2} \cdot {a}^{2} \cdot Δ {z}^{2} \cdot {ω}^{2} \cdot {\hat{η}}^{2}}{{M}^{4}} ) \cdot sin ( \frac{π \cdot Δ z \cdot {λω}^{2} \cdot \hat{η}}{{M}^{4}} ) \qquad ( 23 )$$

Hence, in these configurations, the distances of the focus planes can be determined by numerically solving equation (23).

From an analysis of equation (23), it can be shown that for a medium-sized half angle of divergence α, the following inequality holds true:

$$\frac{{M}^{2}}{\sqrt{2} π \cdot α \cdot \hat{η} \cdot ω} < \vert Δ {z}_{FP} \vert < \frac{{M}^{4}}{2 \cdot λ \cdot \hat{η} \cdot {ω}^{2}} . \qquad ( 24 )$$

The microscope system may be configured to determine the value of Δz_{FP} depending on the size of the illumination spot. The size of the illumination spot may be calculated depending on optical parameters of the illumination beam path and/or depending on parameters characterizing the ametropia of the eye under inspection.

A small illumination spot on the retina can be obtained if the illumination system is configured such that at a minimum cross-section of the illumination beam path, from which the illumination beam path diverges, the following relations holds:

D·sin(β)<M, (25)

wherein D denotes a diameter of the minimum cross-section, and β is an object side angle of divergence of the illumination beam path at the position of the minimum cross-section. The parameter M may have a value of 0.9 millimeters, or a value of 0.5 millimeters, or a value of 0.1 millimeters, or a value of 50 micrometers or a value of 10 micrometers, or a value of 5 micrometers or a value of 2 micrometers.

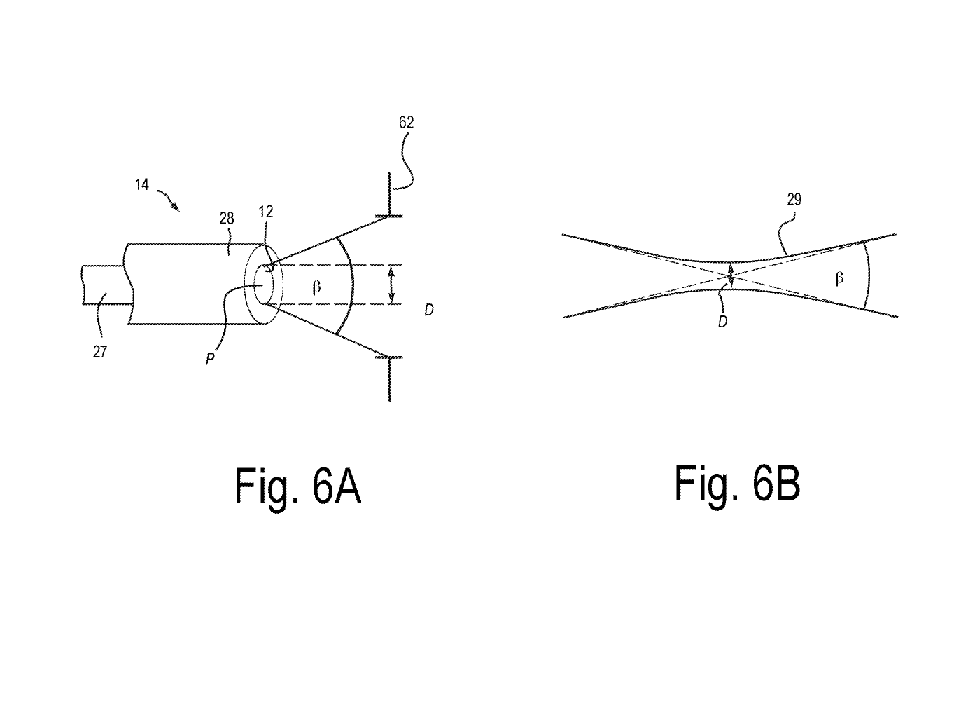

The minimum cross-section may, for example, be a focal point of the illumination beam path or may be a light emitting surface, from which light rays are emitted into an imaging optical system. The light emitting surface may for example be the exit surface 12 (shown in FIG. 2A) of the light guide 14. This is illustrated in detail in FIG. 6A. The light guide 14 includes a light emitting surface 12, a core 27, and a cladding 28. The light emitting surface 12 has a diameter D. From the light emitting surface 12, light rays are emitted which form a maximum angle β. The maximum angle β is the angle of divergence of the illumination beam path at the light emitting surface 12. The value (β/2) may correspond to a numerical aperture of the light guide 14. Alternatively, the angle of divergence β may be limited by an entry pupil 62 of the illumination optics.

Alternatively, as is shown in FIG. 6B, the minimum cross-section may be located in a beam waist of a laser beam 29. The beam waist has a diameter D, which represents the diameter of the minimum cross-section. In this exemplary embodiment, the angle of divergence β is formed by tangents to the far field of the laser beam.

FIGS. 7A and 7B illustrate imaging optics of a microscope system according to a further alternative embodiment. The imaging optics image the object plane OP onto the image plane IP. The imaging optics include two folding mirrors 43 and 44 constituting deflection elements, to obtain a system which is compact in size. The beam path, which is shown in FIGS. 7A and 7B is a first beam path of a first one of two observation channels. Hence, the illustrated first beam path is arranged off-axis. For purposes of simplification of the drawings, the second observation channel is not illustrated. The imaging optics include a first optical component 45 and a second optical component 46. In the illustrated exemplary embodiment, each of the optical components consists of a lens, a lens group and/or a cemented lens. The second optical component 46 is the objective lens of the microscope system. Between the first and the second optical component 45, 46, an aperture stop 47 is arranged in the beam path, configured to limit the numerical aperture.

FIG. 7B illustrates the same illumination optics, as is shown in FIG. 7A, wherein, however, only the principle rays of the first beam path are illustrated. By definition, the principle rays pass through the centre of the aperture 47. The principle rays have been extended to the retina by taking into account the optical power of the eye. In the exemplary embodiment, which is illustrated in FIGS. 7A and 7B, the aperture stop 47 is arranged in the beam path so that the extended principle rays are substantially focused on a point 48 on the retina 6. Thereby, it is possible to obtain an image in the image plane, wherein the anterior section is homogenously illuminated, even if the illumination spot 5 on the retina 6 (shown in FIG. 2) only has a small diameter.

While the disclosure has been described with respect to certain exemplary embodiments thereof, it is evident that many alternatives, modifications and variations will be apparent to those skilled in the art. Accordingly, the exemplary embodiments of the disclosure set forth herein are intended to be illustrative and not limiting in any way. Various changes may be made without departing from the spirit and scope of the present disclosure as defined in the following claims.

1. A microscope system for imaging an object within an eye, wherein the object is a phase object:
wherein the microscope system comprises imaging optics for generating images of an object plane of the imaging optics in an image plane of the imaging optics by using imaging light; wherein the object plane is optically conjugate to the image plane;
wherein the microscope system is configured:
to store focus shift data representing a focus shift, wherein the focus shift is generated by a different effect on the imaging light generated by phase objects as compared to amplitude objects; and
to image the object, depending on the focus shift data.

2. The microscope system according to claim 1, wherein the microscope system is configured to adjust, depending on the focus shift data, a distance between the object plane and the object so that a focus plane of the object is located in the image plane.

3. The microscope system according to claim 2, wherein the adjusting of the distance is further performed depending on an object distance of a reference object, which is an amplitude object.

4. The microscope system according to claim 1, wherein the microscope system is configured to:
generate reference object image data, which represent an image of a reference object, which is an amplitude object; and to
adjust a distance between the object plane and the object, depending on the reference object image data.

5. (canceled)

6. The microscope system according to claim 3, wherein the reference object is configured to at least one of manipulate the object and interact with the object.

7. The microscope system according to claim 1,
wherein the microscope system is configured to determine the focus shift data depending on a spatial frequency to be imaged of a phase object.

8. The microscope system according to claim 1, wherein the microscope system comprises an illumination system configured to generate a light emitting surface within the eye, wherein the microscope system is configured to determine the focus shift data depending on a size of the light emitting surface.

9. The microscope system according to claim 1, further comprising an illumination system, which is configured to generate a red reflex illumination for one or more values of an optical power of the eye.

10. The microscope system according to claim 1, wherein the microscope system comprises an illumination system configured to generate an illumination beam path;
wherein the illumination beam path has a minimum cross-section from which the illumination beam path diverges; wherein the illumination system is configured so that at a position of the minimum cross-section the following relation holds:
D·sin(β)<M;
wherein D is a diameter of the minimum cross-section of the illumination beam path, β is an object side angle of divergence of the illumination beam path at the position of the minimum cross-section, and wherein M has a value of 0.9 mm.

11. The microscope system according to claim 1, wherein the microscope system comprises an illumination system having a single mode light guide.

12. The microscope system according to claim 1, wherein the microscope system is configured to determine an object distance of the object, depending on the focus shift data

13. The microscope system according to claim 1, wherein the microscope system is configured to acquire data, which represent a position of the object plane, at which a sign of an imaging intensity of the object in the image plane, is reversed, relative to a background intensity in the image plane.

14. A method of operating a microscope system for imaging an object, which is imaged by the microscope system as a phase object;
wherein the microscope system comprises imaging optics for generating images of an object plane of the imaging optics in an image plane of the imaging optics by using imaging light; wherein the object plane is optically conjugate to the image plane;
wherein the method comprises:
generating focus shift data representing a focus shift, wherein the focus shift is generated by a different effect on the imaging light generated by phase objects as compared to amplitude objects; and
imaging the object with the microscope system, depending on the determined focus shift data.

15. The method according to claim 14, further comprising:
adjusting, depending on the generated focus shift data, a distance between the object plane and the object so that a focus plane of the object is located in the image plane.

16. The method according to claim 15, wherein the adjusting of the distance is further performed depending on an object distance of a reference object, which is an amplitude object.

17. The method according to claim 14, further comprising:
generating reference object image data, which represent an image of a reference object, which is an amplitude object; and
adjusting a distance between the object plane and the object, depending on the reference object image data.

18. (canceled)

19. The method according to claim 16, wherein the reference object is configured to at least one of manipulate the object and interact with the object.

20. The method according to claim 14, further comprising:
determining the focus shift data depending on a spatial frequency to be imaged of a phase object.

21. The method according to claim 14, further comprising determining the focus shift data depending on a size of a light emitting surface of an illumination system of the microscope system, wherein the illumination system configured to generate the light emitting surface within the eye.

22. Computer program product comprising computer-readable commands, which, when loaded into a memory of a computer and/or a computer network and executed on the computer and/or the computer network, cause the computer and/or the computer network to execute a method of operating a microscope system for imaging an object, which is imaged by the microscope system as a phase object;
wherein the microscope system comprises imaging optics for generating images of an object plane of the imaging optics in an image plane of the imaging optics by using imaging light; wherein the object plane is optically conjugate to the image plane;
wherein the method comprises:
generating focus shift data representing a focus shift, wherein the focus shift is generated by a different effect on the imaging light generated by phase objects as compared to amplitude objects; and
imaging the object with the microscope system, depending on the determined focus shift data.
